(19) United States
(12) Patent Application Publication
(10) Pub. No.: US 2023/0290111 A1
(43) Pub. Date: Sep. 14, 2023

(54) SYSTEMS AND METHODS TO PROCESS ELECTRONIC IMAGES FOR MODEL SELECTION

(71) Applicant: PAIGE.AI, Inc., New York, NY (US)

(72) Inventors: Jeremy Daniel KUNZ, New York, NY (US); Danielle GORTON, Beacon, NY (US); Adam CASSON, New York, NY (US)

(21) Appl. No.: 18/179,871

(22) Filed: Mar. 7, 2023

Related U.S. Application Data
(60) Provisional application No. 63/317,887, filed on Mar. 8, 2022.

(51) Int. Cl. G06V 10/70; G06T 7/00; G06T 5/20; H04N 5/265; G16H 30/20; G16H 30/40

(52) U.S. Cl. CPC G06V 10/70; G06T 5/20; G06T 7/0012; G16H 30/20; G16H 30/40; H04N 5/265; G06T 2207/30024

(57) ABSTRACT

A computer-implemented method for processing electronic medical images, the method including receiving one or more digital medical images of at least one pathology specimen, the pathology specimen being associated with a patient and receiving one or more search criteria. One or more machine learning systems may be determined based on the one or more search criteria. The one or more machine learning systems may be output to a user, wherein outputting the one or more machine learning system includes applying the one or more machine learning systems to the one or more received medical images, and displaying the one or more digital medical images after the machine learning system performed analysis on the digital medical images. A selection from a user may be received, the selection corresponding to a first machine learning system from the one or more machine learning systems. The first machine learning system may be output.

### RELATED APPLICATION

This application claims priority to U.S. Provisional Application No. 63/317,887 filed Mar. 8, 2022, the entire disclosure of which is hereby incorporated herein by reference in its entirety.

### FIELD OF THE DISCLOSURE

Various embodiments of the present disclosure pertain generally to the selection of machine learning (“ML”) models. More specifically, particular embodiments of the present disclosure relate to systems and methods to process electronic images to select models for data visualization.

### BACKGROUND

Companies and research labs have developed a wide range of Artificial Intelligence (“Al”) models in the field of computational biology. Researchers, pathologists, and medical professionals may not be aware of what models are available. Further, even if aware of different models, they may find it difficult to understand the differences between the available models. This may make it difficult for researchers, pathologists, or medical professionals to select a specific model for use based on their situational needs. Additionally, researchers, pathologists, and medical professionals may find it challenging to utilize or combine multiple models at once.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the present disclosure, systems and methods are disclosed for a computer-implemented method for processing electronic medical images, the method including receiving one or more digital medical images of at least one pathology specimen, the pathology specimen being associated with a patient and receiving one or more search criteria. One or more machine learning systems may be determined based on the one or more search criteria. The one or more machine learning systems may be output to a user, wherein outputting the one or more machine learning system includes applying the one or more machine learning systems to the one or more received medical images, and displaying the one or more digital medical images after the machine learning system performed analysis on the digital medical images. A selection from a user may be received, the selection corresponding to a first machine learning system from the one or more machine learning systems. The first machine learning system may be output.

A system for processing electronic digital medical images, the system including: at least one memory storing instructions; and at least one processor configured to execute the instructions to perform operations including: receiving one or more digital medical images of at least one pathology specimen, the pathology specimen being associated with a patient and receiving one or more search criteria. One or more machine learning systems may be determined based on the one or more search criteria. The one or more machine learning systems may be output to a user, wherein outputting the one or more machine learning system includes applying the one or more machine learning systems to the one or more received medical images, and displaying the one or more digital medical images after the machine learning system performed analysis on the digital medical images. A selection from a user may be received, the selection corresponding to a first machine learning system from the one or more machine learning systems. The first machine learning system may be output.

A non-transitory computer-readable medium storing instructions that, when executed by a processor, perform operations processing electronic digital medical images, the operations including: receiving one or more digital medical images of at least one pathology specimen, the pathology specimen being associated with a patient and receiving one or more search criteria. One or more machine learning systems may be determined based on the one or more search criteria. The one or more machine learning systems may be output to a user, wherein outputting the one or more machine learning system includes applying the one or more machine learning systems to the one or more received medical images, and displaying the one or more digital medical images after the machine learning system performed analysis on the digital medical images. A selection from a user may be received, the selection corresponding to a first machine learning system from the one or more machine learning systems. The first machine learning system may be output.

### BRIEF DESCRIPTION OF THE DRAWINGS

The accompanying drawings, which are incorporated in and constitute a part of this specification, illustrate various exemplary embodiments and, together with the description, serve to explain the principles of the disclosed embodiments.

FIG. 1A illustrates an exemplary block diagram of a system and network for processing images to determine an optimal case order, according to techniques presented herein.

FIG. 1B illustrates an exemplary block diagram of a tissue viewing platform according to techniques presented herein.

FIG. 2 is a flow diagram illustrating an exemplary process for using a trained system to determine one or more machine learning models, according to techniques presented herein.

FIG. 3 illustrates an exemplary block diagram of a training module, according to an exemplary embodiment of the present disclosure.

FIG. 4 is a flowchart illustrating an example method for using a trained system to determine one or more machine learning systems based on received criteria, according to one or more exemplary embodiments herein.

FIG. 5 is a flowchart illustrating an example method for using a trained system to determine one or more machine learning systems based on received criteria, according to one or more exemplary embodiments herein.

FIG. 6 is a flowchart illustrating an example method for determining one or more machine learning systems.

FIG. 7 depicts an example of a computing device that may execute techniques presented herein, according to one or more embodiments.

### DESCRIPTION OF THE EMBODIMENTS

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

As used herein, a “machine learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, biases, criteria for forming classifications or clusters, or the like. Deep learning techniques may also be employed. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

The system and techniques described herein may help researchers, pathologists, and professionals compare, visualize, and ensemble artificial intelligence (“Al”) models such as machine learning models within the field of digital pathology. The system may include a system that is capable of receiving and storing AI models developed by different individuals/companies/institutions (e.g. vendors, researchers). The system may be accessible through the internet. The AI models may have different outputs, inputs, and/or visualizations that the system may store and record. The system may store a variety of criteria related to each model such as training size, validation size, European CE Mark approval, U.S. Food and Drug Administration (FDA) approval, FDA approval intended use training and validation data statistical distribution (such as distribution and expected performance on rare cases and conditions), model architecture, model size, model run time (how long it takes the model to run per WSI image size), inputs, outputs, functions performed, and other factors. Functions performed may refer to the type of analysis performed by the machine learning system. Additionally, the system may be able to sort and organize inputted models based on the pathological condition(s) that each model analyzes.

The system described herein may allow users to search for AI models based on various criteria. For example, the system may allow a user to search for an AI model based on a specific disease that an individual (e.g., a pathologist) wishes to analyze. Alternatively, the system may allow a user to search for models that analyze specific biomarkers. Users may be able to further refine searches based on additional criteria such as whether the model will be utilized within a research or a clinical setting. Users may also perform searches that involve applying an initial model to filter their inputs prior to the desired analysis.

After a user searches the system for a model to utilize, the system may further include various methods of visualizing models to users of the system. For example, the system may allow users to upload a whole slide image (“WSI”) of their choice and the system may depict the model as applied to the uploaded WSI. U.S. Non-Provisional App. No. 18/062,677, filed Dec. 7, 2022, is hereby incorporated by reference in its entirety. This application describes example visualization methods that the system described herein may employ to depict the various models within the online store.

The system may further output an AI model ensemble based on a user’s search criteria. The AI model ensemble may be a single outputted model that is a combination of several different AI models meant to provide an optimal predictive AI model. The ensemble may be based on the various criteria saved for each AI model. For example, similar AI models could be compared using the same dataset or multiple datasets where the generalization to multiple datasets is also assessed. The system could predict which AI model or which combination of multiple AI model outputs will be the most accurate output based on all of the inputted search criteria/factors. The system may determine a most accurate AI model using a variety of difference measurements such as Area Under the Curve (AUC), Jaccard index, sensitivity, and/or specificity. For an exemplary search, the system may determine a most accurate output based on performance of the AI model on a specific subpopulation (e.g., female, male, certain morphologies, on rare cancers, etc.).

The system may further analyze and compare (e.g., output) the final chosen diagnosis that all relevant AI models output. The system may keep model-accuracy data, for example data on which models most accurately predict the ultimate diagnosis. Further, the system may record what data was utilized for training and the distribution, characteristics, and scanner type of the data. For example, the system could record that only Leica AT2 data was used in training for a particular AI model.

The system may further have the ability to select combined outputs (overlap outputs) of multiple AI models based off the distribution of training data. For example, a segmentation model that was trained on a Leica scanner AT2 may produce valid outputs, however, the segmentation model might not generalize well to scanners of other manufacturers (e.g., the accuracy may be lower). Thus, the output (e.g., a heat map) from the segmentation model that was trained on a Leica scanner AT2 may be output with a lower confidence on a WSI that was created from a scanner other than a Leica scanner A2. Another AI model trained on the alternative scanner may produce a more accurate output. Generally, the system may lower the confidence in the output whenever there is a mismatch between the scanner model and/or brand of the input and the scanner model(s) and/or brand of the training data. Thus, the overall trained system may then output and overlay both AI models (e.g., as heat maps), while visually indicating (e.g., by markings, highlights, etc.) that the second model’s heat may have a higher degree of accuracy based on the system matching the distribution of the training data more accurately. The user may be able to select an optimal output on a scanner type A and the system may combine AI models/outputs based on the selection/input of the user. For example, a user may select an AI model that is optimized for digital medical images with tissue of an individual that smokes tobacco products. Alternatively, a user may select an AI model to fine tune output based on the brand and/or model of scanner used for training data (e.g., a Hamamatsu, Philips, or Leica scanner).

Multiple labs/users may utilize selected AI models simultaneously. Notifications may be provided to users when new versions of used AI models are available. AI Models may be flagged when any AI models are out of compliance with relevant protocols. For example, an older AI model may be out of compliance with newer guidelines/protocols. Accordingly, AI models may have a guidance version associated with them, and search results may be strongly recommended if an AI model is associated with the current or searched guidance version, weakly recommended if the AI model is in partial compliance with the current or searched guidance version, and not recommended if out of compliance with the current or searched guidance version. Further, the system may automatically provide the updated AI model to the user for analysis of future WSIs. The system may also incorporate the updated AI model into any combination of AI models being used by a user. Users may further be able to visualize and select different versions of the same AI model for use.

The system may include a marketplace for computational AI models. The marketplace may have various vendors and research groups that provide different AI models that are available to users. The system may provide an online store/marketplace that allows users to search for models based on specific criteria. The system may provide visualizations of the different models available for use. The system may further provide an ensemble of different models meant to provide an optimal result based on a user’s search criteria. The ensemble may visualize the data and outputs in a variety of ways.

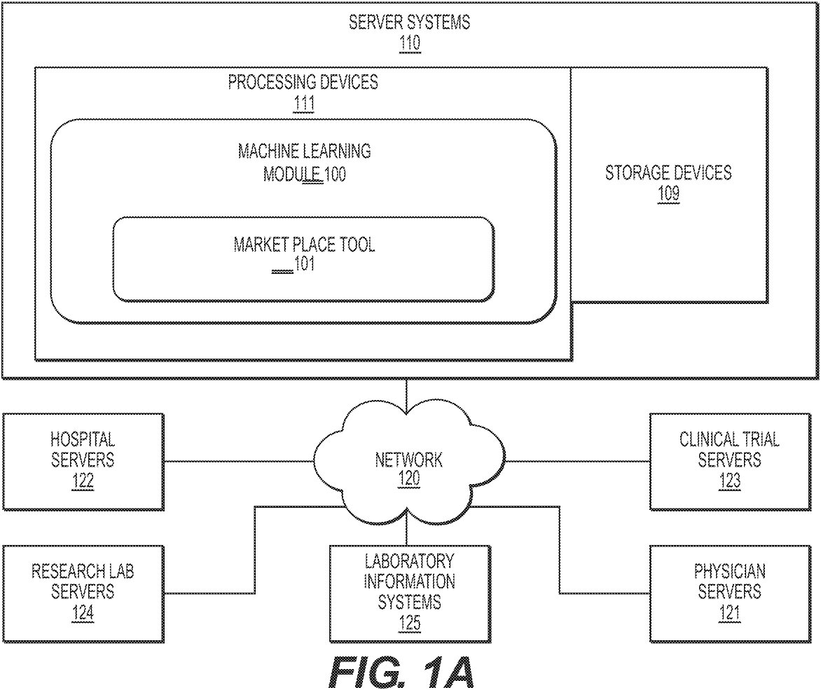

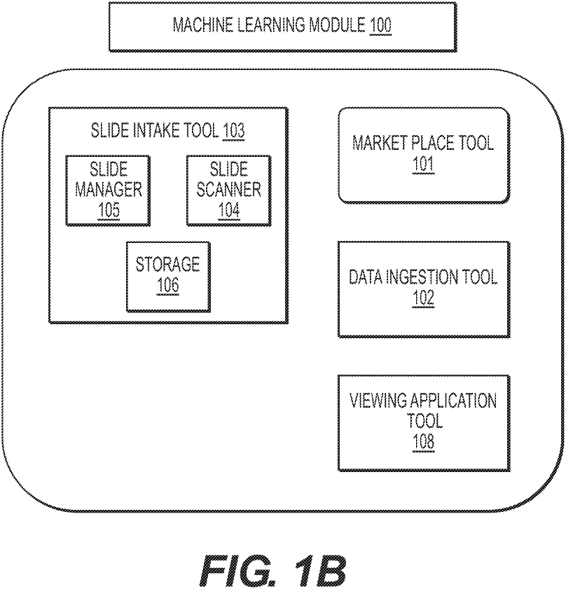

FIG. 1A illustrates a block diagram of a system and network for processing images to determine one or more AI models, according to an exemplary technique of the present disclosure.

Specifically, FIG. 1A illustrates an electronic network 120 that may be connected to servers at hospitals, laboratories, and/or doctors’ offices, etc. For example, physician servers 121, hospital servers 122, clinical trial servers 123, research lab servers 124, and/or laboratory information systems 125, etc., may each be connected to an electronic network 120, such as the Internet, through one or more computers, servers, and/or handheld mobile devices. According to an exemplary embodiment of the present disclosure, the electronic network 120 may also be connected to server systems 110, which may include processing devices 111. One or more of the processing devices 111 may be configured to implement a machine learning module 100, which includes a Marketplace tool 101 for determining one or more AI models based on a user’s search criteria, according to an exemplary technique described herein.

The physician servers 121, hospital servers 122, clinical trial servers 123, research lab servers 124, and/or laboratory information systems 125 may create or otherwise obtain images of one or more patients’ cytology specimen(s), histopathology specimen(s), slide(s) of the cytology specimen(s), digitized images of the slide(s) of the histopathology specimen(s), or any combination thereof. The physician servers 121, hospital servers 122, clinical trial servers 123, research lab servers 124, and/or laboratory information systems 125 may also obtain any combination of patient-specific information, such as age, medical history, cancer treatment history, family history, past biopsy or cytology information, etc. The physician servers 121, hospital servers 122, clinical trial servers 123, research lab servers 124, and/or laboratory information systems 125 may transmit digitized slide images and/or patient-specific information to server systems 110 over the electronic network 120. Server systems 110 may include one or more storage devices 109 for storing images and data received from at least one of the physician servers 121, hospital servers 122, clinical trial servers 123, research lab servers 124, and/or laboratory information systems 125. Server systems 110 may also include processing devices 111 for processing images and data stored in the one or more storage devices 109. Server systems 110 may further include one or more machine learning tool(s) or capabilities. For example, the processing devices 111 may include a machine learning tool for the machine learning module 100, according to one embodiment. Alternatively or in addition, the present disclosure (or portions of the system and methods of the present disclosure) may be performed on a local processing device (e.g., a laptop).

The physician servers 121, hospital servers 122, clinical trial servers 123, research lab servers 124, and/or laboratory information systems 125 refer to systems used by pathologists for reviewing the images of the slides. In hospital settings, tissue type information may be stored in one of the laboratory information systems 125.

FIG. 1B illustrates an exemplary block diagram of a machine learning module 100 for determining one or more alternative machine learning system to apply pertaining to digital pathology image(s), using machine learning.

For example, the machine learning module 100 may include the marketplace tool 101, a data ingestion tool 102, a slide intake tool 103, a slide scanner 104, a slide manager 105, a storage 106, and a viewing application tool 108.

The marketplace tool 101, as described in detail below, refers to a process and system for processing digital pathology slides (e.g., digitized images of slide-mounted histology or cytology specimens) and received AI models, and using machine learning to analyze and determine one or more machine learning systems to output based on a search criteria, according to an exemplary embodiment. The marketplace tool 101 may further apply the determined machine learning systems to one or more digital pathology slides.

The data ingestion tool 102 refers to a process and system for facilitating a transfer of the digital pathology images to the various tools, modules, components, and devices that are used for classifying and processing the digital pathology images, according to an exemplary embodiment.

The slide intake tool 103 refers to a process and system for scanning pathology slides and converting them into a digital form, according to an exemplary embodiment. The slides may be scanned with the slide scanner 104, and the slide manager 105 may process the images on the slides into digitized pathology images and store the digitized images in storage 106.

The viewing application tool 108 refers to a process and system for providing a user (e.g., a pathologist) with specimen property or image property information pertaining to digital pathology image(s), according to an exemplary embodiment. The information may be provided through various output interfaces (e.g., a screen, a monitor, a storage device, and/or a web browser, etc.).

The marketplace tool 101, and one or more of its components, may transmit and/or receive digitized slide images and/or patient information and/or AI models to server systems 110, physician servers 121, hospital servers 122, clinical trial servers 123, research lab servers 124, and/or laboratory information systems 125 over an electronic network 120. Further, server systems 110 may include one or more storage devices 109 for storing images, AI models, and data received from at least one of the marketplace tool 101, the data ingestion tool 102, the slide intake tool 103, the slide scanner 104, the slide manager 105, and the viewing application tool 108. Server systems 110 may also include the processing devices 111 for processing images and data stored in the storage devices 109. Server systems 110 may further include one or more machine learning tool(s) or capabilities, e.g., due to the processing devices 111. Alternatively or in addition, the present disclosure (or portions of the system and methods of the present disclosure) may be performed on a local processing device (e.g., a laptop).

Any of the above devices, tools and modules may be located on a device that may be connected to an electronic network 120, such as the Internet or a cloud service provider, through one or more computers, servers, and/or handheld mobile devices.

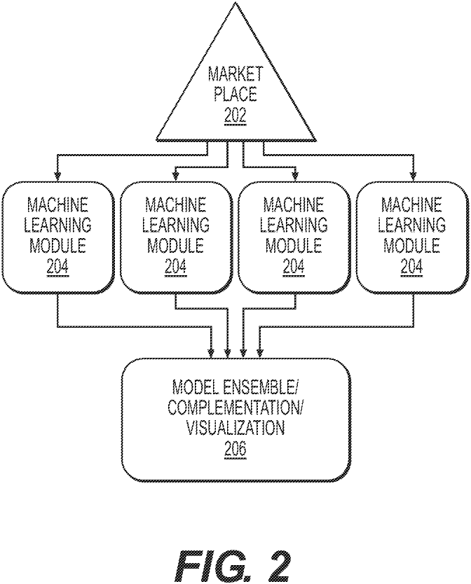

FIG. 2 is a flow diagram illustrating an exemplary process for using a trained system to determine one or more machine learning models, according to techniques presented herein. The trained system may be implemented by the marketplace tool 101 of the machine learning module 100. The marketplace tool 101 may utilize the trained system to determine and apply one or more machine learning system to inputted digital medical images.

The marketplace 202 may receive a collection of machine learning systems. The marketplace 202 may be capable of utilizing the marketplace tool 101 of the machine learning module 100 to determine one or more machine learning systems for output. The determined machine learning systems may be capable of receiving as input digital medical images. The determined machine learning systems may further perform analysis on the received images and output digital medical images and metadata The marketplace 202 may also receive metadata related to the criteria such as: respective training size, validation size, European CE Mark approval, U.S. Food and Drug Administration (FDA) approval, FDA approval intended use training and validation data statistical distribution (such as distribution and expected performance on rare cases and conditions), training data (e.g. which organs and metastatic sites was it trained on), model architecture, model size, model run time (how long it takes the model to run per WSI image size), inputs, outputs, functions performed, and other factors. Additional criteria may include the types of ways the outputs may be displayed (e.g., slide level, part level, segmentation (heat maps), XY-coordinate, etc.). The marketplace 202 may then be capable of organizing and determining machine learning systems to output respective users based on the criteria above.

The marketplace 202 may then be accessed by one or more users as will be discussed further below. The one or more user may search for one or more machine learning systems. The user may perform this search (e.g., a criteria search) based on the criteria discussed above. For example, a user may search for a machine learning system capable of grading cancer of digital medical images.

After one or more search criteria is received by the marketplace 202, the marketplace 202 may determine one or more machine learning systems (e.g., machine learning systems 204) based on the users’ search criteria. The determined machine learning systems 204 may then be displayed/outputted to a user. For example, the determined machine learning systems 204 may each be applied to one or more of the received digital medical images. Once applied, the outputs of the determined machine learning systems 204 may be displayed as separate visuals/screens to one or more users. Further, outputted metadata may be available to one or more users. The user may then select a preferred machine learning system for use learning system) a user may select a machine learning system to download/utilize.

Additionally, information about the determined machine learning modules may be output as metadata describing the model and features. In another example, the marketplace 202 may select an optimal machine learning system for use.

After a machine learning model is selected, the system may output a model ensemble/ complementation, and/or visualization 206 of one or more selected machine learning module or a model ensemble 206. The model ensemble may apply a first determined machine learning system and a second determined machine learning system to the one or more inserted digital medical images. For example, if multiple models have a slide level binary output (e.g., whether cancer is present), the model ensemble may do a majority vote on the binary output and output the majority answer. In another example, if the multiple models output heat maps, then the model ensemble may include an overlay where the intersection of the separately determined heat maps are highlighted/marked to indicate agreement among the one or more models. Additionally, a user may be able to select an option to only output the overlap of the heat maps.

Further, the system may be capable of outputting the selected machine learning system to one or more users. The machine learning system may be implemented locally (e.g., on premises by a pathologist) and/or remotely (e.g., cloud based) wherein the user accesses the selected machine learning system.

The marketplace 202 may be generated based on applying received machine learning systems metadata related to the training size, validation size, European CE Mark approval, U.S. Food and Drug Administration (FDA) approval, inputs, outputs, functions performed, and other factors of each received machine learning system to a machine learning algorithm. The machine learning algorithm may accept, as inputs, the inputs, outputs, and functions of each received machine learning system and implement training using one or more techniques. For example, the generalized machine learning model may be trained in one or more Convolutional Neural Networks (CNN), CNN with multiple-instance learning or multi-label multiple instance learning, Recurrent Neural Networks (RNN), Long-short term memory RNN (LSTM), Gated Recurrent Unit RNN (GRU), graph convolution networks, Transformers or the like or a combination thereof. Convolutional neural networks can directly learn the image feature representations necessary for discriminating among characteristics, which can work extremely well when there are large amounts of data to train on for each specimen, whereas the other methods can be used with either traditional computer vision features, e.g., SURF or SIFT, or with learned embeddings (e.g., descriptors) produced by a trained convolutional neural network, which can yield advantages when there are only small amounts of data to train on. The trained marketplace 202 may be configured to provide one or more machine learning systems (e.g., a machine learning system that detects cancer, or a machine learning system that grades caner) as outputs based on user requested functions of a machine learning system (e.g., the user requests a machine learning system capable of determining the presence of cancer on one or more digital slides). In one example, the marketplace 202 may allow for a user to manually define which types of outputs can be combined/ensembled with each other. The marketplace 202 may be trained by implement training. The marketplace 202 may be configured to suggest a particular one or more machine learning systems based on the overall performance of both systems and compare the AUC for example. The marketplace 202 could further be trained to choose the model which has regulatory approval FDA/CE over non regulatory approved models. Additionally, the marketplace may be optimize AUC in regards to training, validation and test set size when determining a machine learning system.

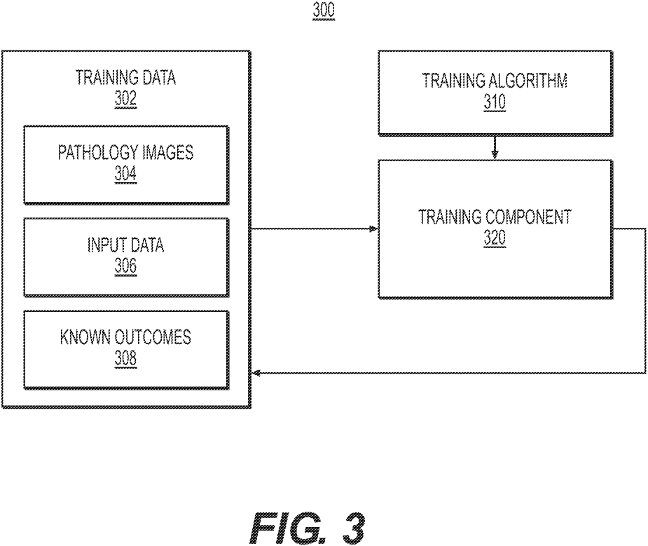

FIG. 3 shows an example training module 300 to train the marketplace 202. As shown in FIG. 3, training data 302 may include one or more of pathology images 304 (e.g., digital representation of biopsied images), input data 306 (e.g., a digital pathology image dataset, various machine learning systems, and the corresponding metadata describing the training size, validation size, European CE Mark approval, U.S. Food and Drug Administration (FDA) approval, inputs, outputs, functions performed, and other factors of the various machine learning systems), and known outcomes 308 (e.g., preferred outputs of machine learning systems) related to the input data 306. The training data 302 and a training algorithm 310 may be provided to a training component 320 that may apply the training data 302 to the training algorithm 310 in order to generate the marketplace 202.

The training dataset including the digital pathology image dataset, various machine learning systems, and the corresponding metadata describing the training size, validation size, European CE Mark approval, U.S. Food and Drug Administration (FDA) approval, inputs, outputs, functions performed of the various machine learning systems may be generated and/or provided by one or more of the systems 110, physician servers 121, hospital servers 122, clinical trial servers 123, research lab servers 124, and/or laboratory information systems 125. Images used for training may come from real sources (e.g., humans, animals, etc.) or may come from synthetic sources (e.g., graphics rendering engines, 3D models, etc.). Examples of digital pathology images may include (a) digitized slides stained with a variety of stains, such as (but not limited to) H&E, Hematoxylin alone, IHC, molecular pathology, etc., and/or (b) digitized tissue samples from a 3D imaging device, such as microCT.

An exemplary embodiment of the system described herein may provide users with the ability to utilize the marketplace system to search for AI models to analyze one or more digital medical images (e.g., WSIs) in order to assist in diagnosis and/or treatment.

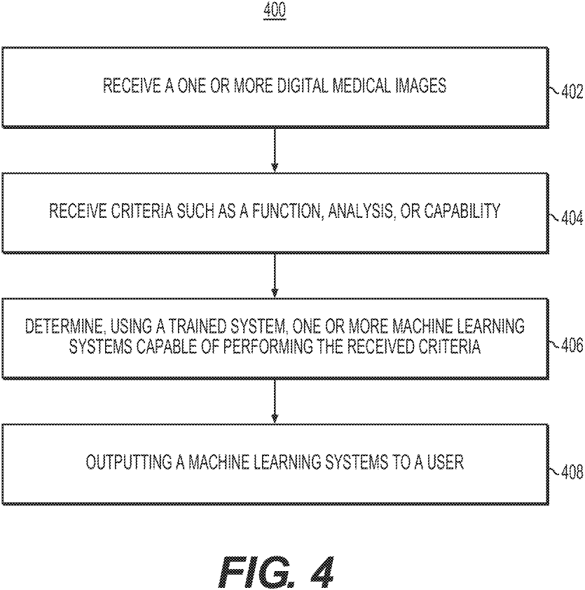

FIG. 4 is a flowchart illustrating an example method for using a trained system to determine one or more machine learning systems based on one or more received criteria, according to one or more exemplary embodiments herein. The exemplary method 400 (e.g., steps 402-408) of FIG. 4 depicts steps that may be performed by, for example, the marketplace 202. These steps may be performed automatically or in response to a request from a user (e.g., a pathologist, a department or laboratory manager, an administrator, etc.). Alternatively, the method 400 may be performed by any computer process system capable of receiving image inputs such as device 700 and capable of storing and executing the marketplace 202.

At step 402, the trained system may receive a plurality of digital medical images of pathology slides associated with pathology cases. Examples of digital medical images may include (a) digitized slides stained with a variety of stains, such as (but not limited to) H&E, Hematoxylin alone, IHC, molecular pathology, etc.; and/or (b) digitized image samples from a 3D imaging device, such as micro-CT. The digital images may be a digital pathology image captured using the slide intake tool 103 of FIG. 1B

At step 404, the trained system may receive criteria (e.g., metadata). The criteria may be for example a function, analysis, or capability of a machine learning system as discussed above. The criteria may be related to the analysis of digital medical images. For example, the search criteria may be a function for determining and grading prostate on a digital medical image to determine a Gleason score.

At step 406, the trained system may use the search criteria from step 404 to determine one or more machine learning systems. The determined machine learning systems may be optimal machine learning systems based on the criteria inputted at step 404 as the criteria may be the task to be solved by the trained system. Determining one or more machine learning systems may include the trained system selecting a set of machine learning systems capable of performing the criteria at step 404. For example, the trained system may suggest for a user a single (e.g., optimal) machine learning system based on the search criteria at step 406. A preferred model may be determined based on a whether each model is up-to-date with latest protocol/guidelines, based on an accuracy determination, based on pricing, and/or based on speed of results.

Alternatively, the trained system may output a set number of (e.g., four) potential machine learning systems and allow a user to select which machine learning system to utilize. Further, when allowing a user to select from the potential machine learning systems, the trained system may be capable of applying each of the potential machine learning systems to one or more of the inputted digital medical images from step 402 and outputting the digital medical images with the applied analysis. This may include outputting digital medical images with digital biomarkers and markings. This may allow the user to see the models applied real time to the inputted digital medical images and help the user determine which model to select/utilize. Further, the trained system may output metadata describing the abilities, function, cost, training, and other aspects of the determined machine learning system. The user may then select a machine learning system based on the initial performed analysis as well as the outputted metadata.

By examining multiple models performing the same or similar function, the user may have increased confidence in the output. During visualization of the determined machine learning systems, the trained system may provide/output an overlap, such as an elevation overlap, of multiple regions of interest/salient regions, each region of interest corresponding to the output of one model. Alternatively, the overlap may include a heat map, level of confidence map, and/or color gradient map. The visualization may allow a user to contrast similar Al models quickly. Areas where multiple models overlap may be indicated as having a high confidence value. For example, if three prostate cancer models partially overlap, the system may indicate/designate that the overlapping region corresponds to prostate cancer with high confidence. Regions with a subset of models partially overlapping may be designated as having intermediate confidence. Regions with only a single model, and no other models, may be designated as a low confidence region. Regions with different confidence values may be colored differently, may be visually elevated or zoomed differently, or otherwise visually indicated differently. For example, a high confidence region may be highlighted in a predetermined color, and/or may appear visually raised to the user. The confidence level output may be displayed separately or with the displayed determined machine learning levels. The confidence level may be output as a heat map coloring grade (e.g., an overlap layer). A user may be able to select which model or models to incorporate based on the outputted confidence levels.

At step 408, the trained system may output the one or more determined machine learning systems to one or more users. Outputting the one or more determined machine learning system may include exporting the determined machine learning system from either the storage devices 109, clinical trial server 123, physician servers 121, laboratory information system 125, research lab servers 124, hospital servers 122, or from an external network. Further, outputting the one or more determined machine learning systems may include applying the determined machine learning system to the one or more digital medical images from step 402 and outputting the analyzed digital images to a user.

In an embodiment, the trained system may also provide users with the ability to utilize the marketplace system to search for Al models to analyze one or more digital medical images (e.g., WSls) in order to assist in diagnosis and/or treatment. In this embodiment, the user may be able to search for Al models based on a specific type of medical problem. The system may then select related models that highlight different aspects related to the same type of problem searched. The system may thus provide models that analyze different aspects of a WSI that are all related to the same type of problem. For example, a breast cancer model may be used along with a calcification model when an inserted search criterion is “breast cancer diagnosis.” In another example, a first model may be used to determine whether tissue is cancerous, malignant or benign. This may then be used with a second model that outputs a Gleason grade. In another example, when analyzing prostate needle core biopsy length, multiple estimation models may be applied and a median length may be determined based on averages of all potential models. In another example, a first model may be selected to determine whether cancer is present on a tissue and a second model be selected to provide additional analysis such as Triple-Negative Breast Cancer (TNBC) prediction(s) (including levels of HER2, estrogen receptor (ER), and/or progesterone receptor (PR)).

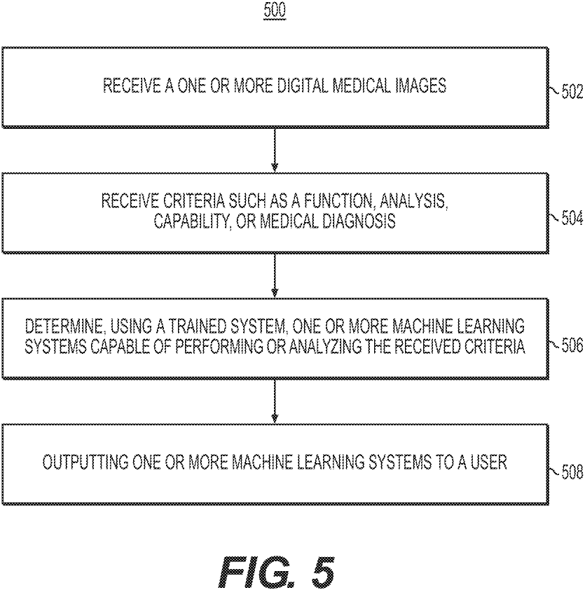

FIG. 5 is a flowchart illustrating an example method for using a trained system to determine one or more machine learning systems based on one or more received criteria, according to one or more exemplary embodiments herein. The exemplary method 500 (e.g., steps 502-508) of FIG. 5 depicts steps that may be performed by, for example, the marketplace 202. These steps may be performed automatically or in response to a request from a user (e.g., a pathologist, a department or laboratory manager, an administrator, etc.). Alternatively, the method 500 may be performed by any computer process system capable of receiving image inputs such as device 700 and capable of storing and executing the marketplace 202.

At step 502, the trained system may receive a plurality of digital medical images of pathology slides associated with pathology cases. Examples of digital medical images may include (a) digitized slides stained with a variety of stains, such as (but not limited to) H&E, Hematoxylin alone, IHC, molecular pathology, etc.; and/or (b) digitized image samples from a 3D imaging device, such as micro-CT. The digital image may be a digital pathology image captured using the slide intake tool 103 of FIG. 1B

At step 504, the trained system may receive criteria (e.g., metadata). The criteria may be for example a function, analysis, or capability of a machine learning system. The criteria may further be a particular medical diagnosis or a particular medical problem. Exemplary medical diagnosis could be the presence of one or more types of cancer.

At step 506, the trained system may use the search criteria from step 504 to determine one or more machine learning systems. The determined machine learning systems may be capable of performing and/or analyzing the criteria input at step 504 on the inputted slides from step 502. Determining one or more machine learning systems may include the trained system selecting a set of machine learning systems capable of performing the criteria at step 504. The set of machine learning systems may be based on the trained system determining all Al models that include the search criteria. Determining one or more sets of machine learning system may include the trained system filtering through its library to determine machine learning models that may analyze different aspects of the problem selected For example, the trained system may suggest for a user a set of preferred (e.g., optimal) machine learning systems based on the search criteria at step 506. The preferred model may be determined based on a whether each model is up-to-date with latest protocol/guidelines, based on an accuracy determination, based on pricing, and/or based on speed of results. The preferred models may be capable of analyzing distinct aspects of a particular medical issue.

Alternatively, the trained system may output multiple sets of potential machine learning systems and allow a user to select which machine learning system to utilize. Each set of machine learning systems may correspond to a different aspect of the medical issue selected as criteria. Further, when allowing a user to select from the potential machine learning systems, the trained system may be capable of applying each of the potential machine learning systems to one or more of the inputted digital medical images from step 502 and outputting the digital medical images with the applied analysis. This may include outputting digital medical images with digital biomarkers and marking. This may allow the user to see the models applied real time to the inputted digital medical images and help the user determine which model to utilize/select. The user may then select a machine learning systems based on the initial performed analysis.

At step 508, the trained system may output the one or more determined machine learning systems to one or more users. Outputting the one or more determined machine learning system may include exporting the determined machine learning system from either the storage devices 109, clinical trial server 123, physician servers 121, laboratory information system 125, research lab servers 124, hospital servers 122, or from an external network. Further, outputting the one or more determined machine learning systems may include applying the determined machine learning system to the one or more digital medical images from step 502 and outputting the analyzed digital images to a user. In another embodiment, the trained system (e.g., marketplace 202) of FIG. 5 may be used to analyze digital medical images that have multiple tissue types. The trained system may filter/search for machine leaning models to analyze one or more digital medical images (e.g., WSls) in order to assist in diagnosis and/or treatment of inserted digital medical images with multiple tissue types. In this embodiment, the system may determine, prior to step 506, what tissue types are present from the inserted digital medical images at step 502. The trained system may determine tissue type by receiving metadata indicating the tissue type of each received digital medial image. In another example, the system may utilize a trained machine learning system to determine what types of tissue are located on the digital medical images. Once the tissue types have been determined, at step 506, the trained system may determine machine learning systems capable of analyzing the digital medical images for each tissue type.

An example may include the trained system determining different machine learning models for cancer analysis when receiving digital medical images of prostate and/or bladder tissue. In this scenario, it may be necessary to have different models analyze each type of tissue to accurately identify cancerous regions. Additional tissue types that may be included in a received digital image include, but are not limited, to pancreas, duodenum, bile duct, and distal stomach tissue, although any tissue type may be used.

In another embodiment, the trained system (e.g., marketplace 202) of FIG. 5 may be used to analyze digital medical images and determine an initial one or more machine learning system, followed by a second one or more machine learning systems. In this embodiment, the trained system may first, prior to step 506, determine and utilize an area/tile/tissue filter model that may reduce the scope or size of the inserted digital medical images from step 502. Alternatively, the first trained system may first, prior to step 506, determine and utilize a first machine learning system to remove a layer/factor from the digital medical images. For example, a first machine learning system may remove the pixels of benign stroma tissue from the received digital medical image. The initial machine learning system may be configured to remove a layer of information from the received digital medical images. This initial filter may lead to the second model being more accurate. Next, during step 506, the remaining area of the inserted digital medical images may be analyzed using one or more of the determined machine learning models. These determined machine learning models may perform clinical or diagnostic analysis. In one example, the trained system may select the first filter model automatically. This embodiment may lead to the second model being applied to a smaller area of a provided WSI. Optionally, the system may select the first filter model automatically and allow a user to select the second model. This embodiment may lead to the second model being applied to a WSI that has had certain factors removed.

For example, a user may wish to analyze a digital medical image to grade cancer. The user may input the type of cancer to be graded and a WSI into the system. The system may choose a first model that first is used to detect a region of the WSI with cancer. The system may then determine one or more machine learning models to perform cancer grading only on the remaining non-filtered regions of the digital medical images. This may lead to more accurate cancer grading. In one technique, the digital medical images may be divided into tiles/sub-images, and only tiles that the first model determines contains cancerous tissue beyond a threshold might be provided to the second model that performs cancer grading.

In another example, the trained system may be utilized within the field of statistical confounding factors. If certain diseases contain confounding factors that co-occur with a disease, the trained system may first determine a machine learning system configured to remove the confounding factor prior to further machine learning systems being applied.

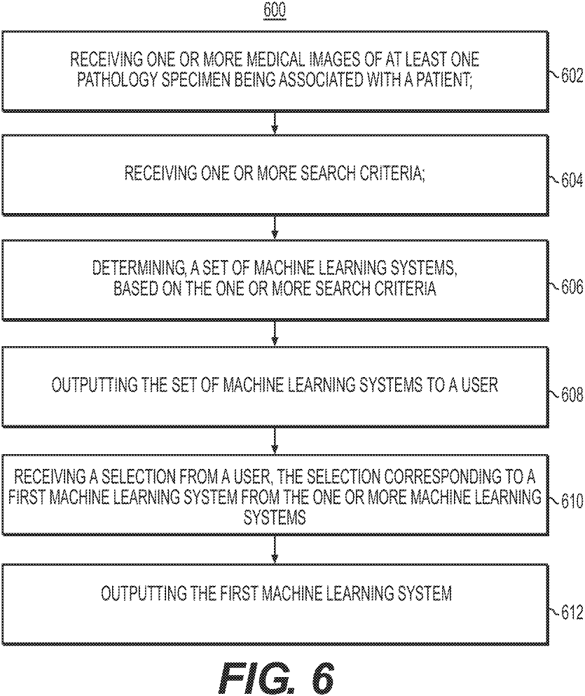

FIG. 6 is a flowchart 600 illustrating an example method for determining one or more machine learning systems. At step 602, one or more digital medical images of at least one pathology specimen may be received, the pathology specimen being associated with a patient. At step 604, one or more search criteria may be received.

At step 606, one or more machine learning systems may be determined based on the one or more search criteria.

At step 608, the one or more machine learning systems may be output to a user, wherein outputting the one or more machine learning system includes the one or more machine learning systems being applied to the one or more received medical images and the one or more digital medical images being displayed after the machine learning system performed analysis on the digital medical images.

At step 610, a selection from a user may be received, the selection corresponding to a first machine learning system from the one or more machine learning systems.

At step 612, the first machine learning system may be output.

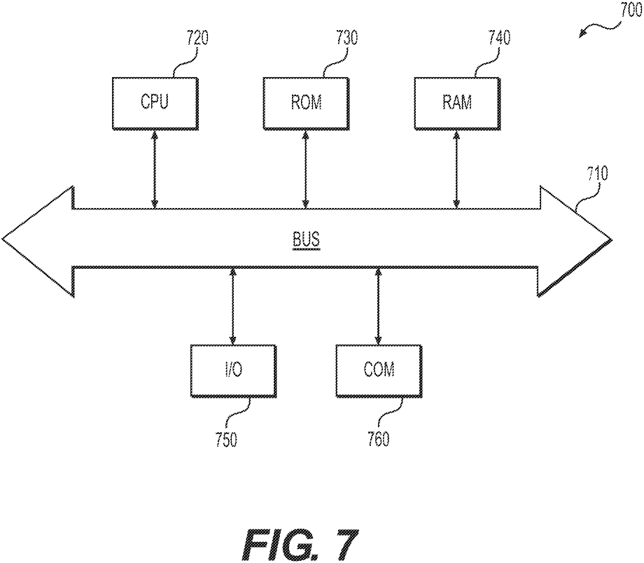

As shown in FIG. 7, device 700 may include a central processing unit (CPU) 720. CPU 720 may be any type of processor device including, for example, any type of special purpose or a general-purpose microprocessor device. As will be appreciated by persons skilled in the relevant art, CPU 720 also may be a single processor in a multi-core/multiprocessor system, such system operating alone, or in a cluster of computing devices operating in a cluster or server farm. CPU 720 may be connected to a data communication infrastructure 710, for example a bus, message queue, network, or multi-core message-passing scheme.

Device 700 may also include a main memory 740, for example, random access memory (RAM), and also may include a secondary memory 730. Secondary memory 730, for example a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 730 may include similar means for allowing computer programs or other instructions to be loaded into device 700. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 700.

Device 700 also may include a communications interface (“COM”) 760. Communications interface 760 allows software and data to be transferred between device 700 and external devices. Communications interface 760 may include a modem, a network interface (such as an Ethernet card), a communications port, a PCMCIA slot and card, or the like. Software and data transferred via communications interface 760 may be in the form of signals, which may be electronic, electromagnetic, optical or other signals capable of being received by communications interface 760. These signals may be provided to communications interface 760 via a communications path of device 700, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 700 may also include input and output ports 750 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components and/or modules may be implemented in software, hardware, or a combination of software and/or hardware.

The tools, modules, and/or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

The foregoing general description is exemplary and explanatory only, and not restrictive of the disclosure. Other embodiments may be apparent to those skilled in the art from consideration of the specification and practice of the invention disclosed herein. It is intended that the specification and examples be considered as exemplary only.

What is claimed is:

1. A computer-implemented method for processing electronic medical images comprising:
receiving one or more digital medical images of at least one pathology specimen, the pathology specimen being associated with a patient;
receiving one or more search criteria;
determining, one or more machine learning systems, based on the one or more search criteria;
outputting the one or more machine learning systems to a user, wherein outputting the one or more machine learning system includes:
applying the one or more machine learning systems to the one or more received medical images, and
displaying the one or more digital medical images after the machine learning system performed analysis on the digital medical images;
receiving a selection from a user, the selection corresponding to a first machine learning system from the one or more machine learning systems; and
outputting the first machine learning system.

2. The method of claim 1, wherein outputting the first machine learning system further includes:
inserting the one or more digital medical images into the first machine learning system;
applying the first machine learning system to the inserted digital medical images to generate processed medical images; and
outputting the generated processed medical images.

3. The method of claim 1, wherein the display of the one or more images includes a heat map, level of confidence map, and/or color gradient map of the search criteria.

4. The method of claim 1, wherein the display of the one or more images includes a heat map overlay of the one or more medical images indicating a confidence value, the confidence value representing a similarity of results between the machine learning systems.

5. The method of claim 1, further including:
suggesting a particular machine learning system of the one or more machine learning systems based on the search criteria.

6. The method of claim 1, further including:
applying a first machine learning system to the received one or more medical images prior to outputting the machine learning systems to a user.

7. The method of claim 6, wherein the first machine learning system applies an initial filter to the one or more medical images, the initial filter to determine an area of tissues displayed in the one or more medical images.

8. The method of claim 1, wherein the search criteria is a training size, validation size, European CE Mark approval, U.S. Food and Drug Administration (FDA) approval, inputs, outputs, or function.

9. The method of claim 1, wherein the search criteria is a medical diagnosis.

10. A system for processing electronic digital medical images, the system comprising:
at least one memory storing instructions; and
at least one processor configured to execute the instructions to perform operations comprising:
receiving one or more digital medical images of at least one pathology specimen, the pathology specimen being associated with a patient;
receiving one or more search criteria;
determining, one or more machine learning systems, based on the one or more search criteria;
outputting the one or more machine learning systems to a user, wherein outputting the one or more machine learning system includes:
applying the one or more machine learning systems to the one or more received medical images, and
displaying the one or more digital medical images after the machine learning system performed analysis on the digital medical images;
receiving a selection from a user, the selection corresponding to a first machine learning system from the one or more machine learning systems; and
outputting the first machine learning system.

11. The system of claim 10, wherein outputting the first machine learning system further includes:
inserting the one or more digital medical images into the first machine learning system;
applying the first machine learning system to the inserted digital medical images to generate processed medical images; and
outputting the generated processed medical images.

12. The system of claim 10, wherein the display of the one or more images includes a heat map, level of confidence map, and/or color gradient map of the search criteria.

13. The system of claim 10, wherein the display of the one or more images includes a heat map overlay of the one or more medical images indicating a confidence value, the confidence value representing a similarity of results between the machine learning systems.

14. The system of claim 10, further including:
suggesting a particular machine learning system of the one or more machine learning systems based on the search criteria.

15. The system of claim 10, further including:
applying a first machine learning system to the received one or more medical images prior to outputting the machine learning systems to a user.

16. The system of claim 10, wherein the first machine learning system applies an initial filter to the one or more medical images, the initial filter to determine an area of tissues displayed in the one or more medical images.

17. The system of claim 10, wherein the search criteria is a training size, validation size, European CE Mark approval, U.S. Food and Drug Administration (FDA) approval, inputs, outputs, or function.

18. The system of claim 10, wherein the search criteria is a medical diagnosis.

19. A non-transitory computer-readable medium storing instructions that, when executed by a processor, perform operations processing electronic digital medical images, the operations comprising:
receiving one or more digital medical images of at least one pathology specimen, the pathology specimen being associated with a patient;
receiving one or more search criteria;
determining, one or more machine learning systems, based on the one or more search criteria;
outputting the one or more machine learning systems to a user, wherein outputting the one or more machine learning system includes:
applying the one or more machine learning systems to the one or more received medical images, and
displaying the one or more digital medical images after the machine learning system performed analysis on the digital medical images;
receiving a selection from a user, the selection corresponding to a first machine learning system from the one or more machine learning systems; and
outputting the first machine learning system.

20. The computer-readable medium of claim 19, wherein the display of the one or more images includes a heat map, level of confidence map, and/or color gradient map of the search criteria.
